(19) United States
(12) Patent Application Publication
(10) Pub. No.: US 2016/0278415 A1
(43) Pub. Date: Sep. 29, 2016

(54) Food Compositions Containing All Essential Nutrients

(71) Applicant: Nutrient Foods, LLC, Reno, NV (US)

(72) Inventor: Charles H. Marsland, Reno, NV (US)

(21) Appl. No.: 15/010,090

(22) Filed: Jan. 29, 2016

Related U.S. Application Data
(60) Provisional application No. 62/109,911, filed on Jan. 30, 2015.

(51) Int. Cl. A01N 59/26; A61K 31/198; A61K 31/405; A61K 31/4172; A61K 31/07; A61K 31/375; A61K 33/26; A61K 31/59; A61K 31/355; A61K 31/122; A61K 31/51; A61K 31/525; A61K 31/455; A61K 31/675; A61K 31/519; A61K 31/714; A61K 31/4188; A61K 33/42; A61K 33/06; A61K 33/30; A61K 33/04; A61K 33/34; A61K 33/32; A61K 33/24; A61K 31/14; A61K 31/202

(52) U.S. Cl. CPC A23L 1/30; A61K 31/14; A61K 31/202; A61K 31/198; A61K 31/405; A61K 31/4172; A61K 31/07; A61K 31/375; A61K 33/26; A61K 31/59; A61K 31/355; A61K 31/122; A61K 31/51; A61K 31/525; A61K 31/455; A61K 31/675; A61K 31/519; A61K 31/714; A61K 31/4188; A61K 33/42; A61K 33/06; A61K 33/30; A61K 33/04; A61K 33/34; A61K 33/32; A61K 33/24; A23L 1/3051; A23L 1/303; A23L 1/302; A23L 1/304; A23L 1/3008; A23L 1/308; A23V 2002/00

(57) ABSTRACT

A food composition that includes at least the following essential nutrients: water, choline, omega-3, omega-6, leucine, isoleucine, lysine, methionine, cysteine, phenylalanine, tyrosine, tryptophan, threonine, valine, histidine, arginine; vitamin A; vitamin C; calcium; iron; vitamin D; vitamin E; vitamin K; thiamin; riboflavin; niacin; vitamin B6; folate; vitamin B12; biotin; pantothenic acid; phosphorus; iodine; magnesium; zinc; selenium; copper; manganese; chromium; molybdenum; and chloride.

### CROSS-REFERENCE TO RELATED APPLICATION

This application claims priority of Provisional Patent Application 62/109,911, filed on Jan. 30, 2015, the disclosure of which is incorporated herein by reference.

### FIELD

This disclosure relates to nutritional food compositions.

### BACKGROUND

The nutritional literature indicates broad agreement as to which nutrients are essential and how much of each of them people need daily. Despite this knowledge, people eat a poor diet, which is low in essential nutrients, according to the 2010 U.S. Dietary Guidelines. Chronic, low consumption of essential nutrients leads to: suboptimal physical and impaired mental development in children, and increased oxidative stress leading to an increased risk of chronic conditions of aging in adults (e.g., obesity, heart disease, and diabetes). To overcome low intakes, some people take dietary supplements containing essential nutrients. Most commercially available supplements for children and adults do not contain all of the essential nutrients, do not contain enough of them, or both of these. Some contain more than enough (i.e., >100% Daily Value [DV]). As such, most supplements do not address the problem of providing adequate and appropriate amounts of essential nutrients; and using pills to obtain essential nutrients, rather than getting them through foods, does not foster good eating habits.

There are known nutrients, which are essential for life. There is awareness from observational studies that certain diseases can be prevented by eating certain foods.

### DEFINITIONS OF ESSENTIAL NUTRIENTS

Following are accepted definitions of essential nutrients:
The substance is required in the diet for growth, health, and survival.
Its absence from the diet or inadequate intake results in characteristic signs of deficiency disease and, ultimately, death.
Growth failure and characteristic signs of deficiencies are prevented only by the nutrient or a specific precursor of it, not by other substances.
Below some critical level of intake of the nutrient, growth response and severity of sign of deficiency are proportional to the amount consumed.
The substance is not synthesized in the body and is, therefore, required to be ingested throughout life.

### Nutrition Labeling.

Packaged foods are labeled, where essential nutrients are expressed as a percentage of the Daily Value (% DV). The 100% DV depicts what people need every day. These are for children aged 4 years of age and older, and adults. There are other nutrients that don't garner a % DV, but are nevertheless important.

Listed below in Table 1 are the essential nutrients as expressed by the FDA, along with the amount equal to 100% DV.

TABLE 1

| | | |
|---|---|---|
| Total fat | 65 | g |
| Saturated fat | 20 | g |
| Cholesterol | 300 | mg |
| Sodium | 2,400 | mg |
| Potassium | 3,500 | mg |
| Total carbohydrate | 300 | g |
| Dietary fiber | 25 | g |
| Vitamin A | 5,000 | IU |
| Vitamin C | 60 | mg |
| Calcium | 1,000 | mg |
| Iron | 18 | mg |
| Vitamin D | 400 | IU |
| Vitamin E | 30 | IU |
| Vitamin K | 80 | mcg |
| Thiamin | 1.5 | mg |
| Riboflavin | 1.7 | mg |
| Niacin | 20 | mg |
| Vitamin B6 | 2 | mg |
| Folate | 400 | mcg |
| Vitamin B12 | 6 | mcg |
| Biotin | 300 | mcg |
| Pantothenic acid | 10 | mg |
| Phosphorus | 1,000 | mg |
| Iodine | 150 | mcg |
| Magnesium | 400 | mg |
| Zinc | 15 | mg |
| Selenium | 70 | mcg |
| Copper | 2 | mg |
| Manganese | 2 | mg |
| Chromium | 120 | mcg |
| Molybdenum | 75 | mcg |
| Chloride | 3,400 | mg |

The main reason that people don't get all of the essential nutrients and don't get them at the right amounts is that people eat the wrong foods. Essential nutrient-rich foods include fruits, vegetables, whole grains, and dairy products. Table 2 lists foods which are rich in nutrients but which are typically not consumed in sufficient quantities so as to supply 100% of the DV of each essential nutrient. The percentage values in the table are the percentage of the 100% DV amount which is consumed by the average person per day.

TABLE 2

| | |
|---|---|
| Whole grains | (15%) |
| Vegetables | (59%) |
| Fruits | (42%) |
| Dairy | (52%) |
| Seafood | (44%) |
| Oils | (61%) |

Also, people often eat too much of the wrong foods (e.g., salt, sugar, saturated fats), which increases the risk of chronic diseases like obesity, diabetes, and heart disease. Table 3 lists examples of these “wrong foods” which are often eaten in excess. The percentage values in the table are the percentage of the 100% DV amount which is consumed by the average person per day.

TABLE 3

| | |
|---|---|
| Calories from solid fats, added sugars | (280%) |
| Refined grains | (200%) |
| Sodium | (149%) |
| Saturated fats | (110%) |

There is no evidence that the diet will improve. Researchers at the Harvard School of Public Health do not embrace the 2010 U.S. Dietary Guidelines (U.S. Department of Agriculture and U.S. Department of Health and Human Services. Dietary Guidelines for Americans 2010, 7^{th} Edition, Washington D.C.: U.S. Government Printing Office, December 2010) and created their own measure to assess how well the population eats (Wang DD. JAMA Intern Med 2014; September 1). Both measures found much the same thing—Americans cat a poor diet. Between 1999 and 2010 one score of diet quality increased slightly, but half of the improvement was related to a decrease in trans fat intake. These were removed (or banned) from most foods during this period, so the change was expected. People also ate a bit more foods containing essential nutrients (e.g., fruit, whole grains, and nuts and legumes), but overall, the American diet is still considered poor according to these investigators.

More Confirmation that People Don't Get all of their Essential Nutrients

Most people (adults and children) don't get all of the essential nutrients at 100% DV every day. Charts A and B of FIG. 1 set out the percentage of the population with vitamin (A) and mineral (B) intakes below the estimated average requirement (EAR) for individuals aged ≧2 years (data from NHANES 2003-2006; n=16,110). Usual intakes from foods (naturally occurring and that from naturally occurring plus added via enrichment and/or fortification) and dietary supplements were estimated by using the National Cancer Institute method with 2 d of reported intake. (EAR, estimated average requirement; Vit, vitamin; Ribo, riboflavin.)

From FIG. 1, it is clear that food alone does not regularly assure that all essential nutrient needs are met in adults and children ≧2 years of age. And, the failure to get 100% DV of the essential nutrients is even true for those who eat regular food and use fortified foods (like cereal containing vitamins and minerals), and for those who eat regular food, use fortified foods and take supplements.

Children have chronic low intakes of essential nutrients, just like adults. Those who just consume foods have suboptimal intakes of many nutrients. Children who consume foods and fortified foods have some improvement, and those who consume foods, fortified foods, and take dietary supplements experience even greater increases in the intake of essential nutrients. However, many are still consumed at suboptimal amounts. Charts A and B of FIG. 2 set out the percentage of the population with usual intake below the EAR. Subgroups shown had the lowest (A, children aged 2 to 8 years) and highest (B, females aged 14 to 18 years) prevalence of inadequate intakes among the total population aged 2 to 18 years.

### Consequences of not Eating Enough Essential Nutrients

Adults.

Many people fail to meet all of their micronutrient needs each day. It has been postulated that chronic, low micronutrient intake may accelerate some diseases of aging (e.g., cancer, heart disease, and diabetes), and that regular intake of all essential nutrients can reduce these slow aging and reduce disease risk.

Americans consume a lot of micronutrient-poor foods, which are usually tasty, inexpensive, and calorically dense. This leads to a chronic inadequate consumption of essential micronutrients. Significant metabolic disruptions occur as a result, leading to premature aging and increased risk of chronic disease. Table 4 sets forth examples of what happens with inadequate micronutrient intakes of specific nutrients over time. The proposed mechanisms for how the lack of essential nutrients affect health is related to: (1) direct tissue and cell damage by oxidative free radicals, and (2) chromosomal breaks (DNA damage).

TABLE 4

| | Consequences of inadequate intake over |
|---|---|
| Micronutrient | time |
| Antioxidants like | Free radical damage, especially to the |
| vitamin C and E | mitochondria |
| | Accelerated aging |
| | Impaired cognition |
| Magnesium | Colon cancer |
| | High blood pressure |
| | Osteoporosis |
| | Diabetes and metabolic syndrome |
| Vitamin D | Cancer of the breast, colon, prostate, |
| | pancreas |
| | Heart disease |
| Calcium | Diabetes |
| Potassium | Heart disease |
| Omega-3s | Melanoma and other cancers |
| | Impaired cognition |
| Vitamin B12 | Cognition |
| Thiamine | Cognitive impairment |
| | Diabetes |
| Iron | Diminished immune function |
| | Neuromuscular abnormalities |

In the absence of adequate micronutrients, the body may shunt the few that are available for ATP synthesis (energy for the body, which is needed immediately). This action would be done at the expense of not fixing DNA damage or reducing oxidative stress to blood cells and organ tissues. This is a mechanism for allocating scare micronutrients to favor short-term survival at the expense of long-term health. The need for meeting all of the micronutrient needs on a regular basis is thus quite significant.

Children.

Children eating foods have suboptimal intakes of essential nutrients, especially for vitamins A, D, E, folate, and calcium. Fortification noticeably increases the intakes of iron, and most other nutrients except vitamin E. With each additional source of nutrients (going from food alone, to food plus fortified/enriched foods, to food plus fortified/enriched foods and dietary supplements), the percentage of the total population with suboptimal nutrient intakes was reduced. Few children consume essential nutrients in excess of the upper limit of safety. The group most at risk is those aged 2 to 8 years, and the risk is limited to folic acid, niacin, and zinc. It is a challenge especially in children to assure that they consume the target nutrient goals. Technical challenges, including taste, mass, or stability issues, present barriers to the addition of some shortfall nutrients; therefore fortification is not a panacea.

Hidden hunger is a nutritional deficiency that occurs in the presence of an otherwise nutritionally or calorically appropriate diet. Hidden hunger has been identified as a major public health issue as individuals with borderline micronutrient deficiencies do not develop classic signs and symptoms of over deficiencies. However, if not identified and managed appropriately, affected individuals will suffer from long-term consequences, such as stunted growth and varying degrees of neurocognitive deficit.

### SUMMARY

This disclosure features a line of foods that are made from wholesome ingredients that are chosen for nutrition, taste, and mouth-feel. The foods are, as needed, enriched with sources of essential nutrients, so that each food composition provides at least about 50% DV for each essential nutrient. Preferably, but not necessarily, each also provides at least about 25 grams protein. Each serving of the subject foods complies with healthy eating criteria for fat, sugar, and salt, and preferably provides close to one-third of the daily allotment of each. The foods are meant to replace all or part of a meal. The foods are appropriate for children aged four years and older, and adults. People can for example consume one or two servings of these meals a day and eat other foods for the rest of their dietary needs.

Table 5 sets forth the 43 “essential nutrients” of the foods of the present disclosure. The disclosed food compositions each include at least some amount of each of these 43 essential nutrients.

TABLE 5

Water
Omega-3 fatty acids
Omega-6 fatty acids
Sodium
Potassium
Essential amino acids (12): leucine,
isoleucine, lysine, methionine, cysteine,
phenylalanine, tyrosine, tryptophan,
threonine, valine, histidine, arginine
Vitamin A
Vitamin C
Calcium
Iron
Vitamin D
Vitamin E
Vitamin K
Thiamin
Riboflavin
Niacin
Choline
Vitamin B6
Folate
Vitamin B12
Biotin
Pantothenic acid
Phosphorus
Iodine
Magnesium
Zinc
Selenium
Copper
Manganese
Chromium
Molybdenum
Chloride

### BRIEF DESCRIPTION OF THE DRAWINGS

FIG. 1 includes two charts that illustrate the percentage of the population with vitamin (A) and mineral (B) intakes below the EAR for individuals aged 2 years.

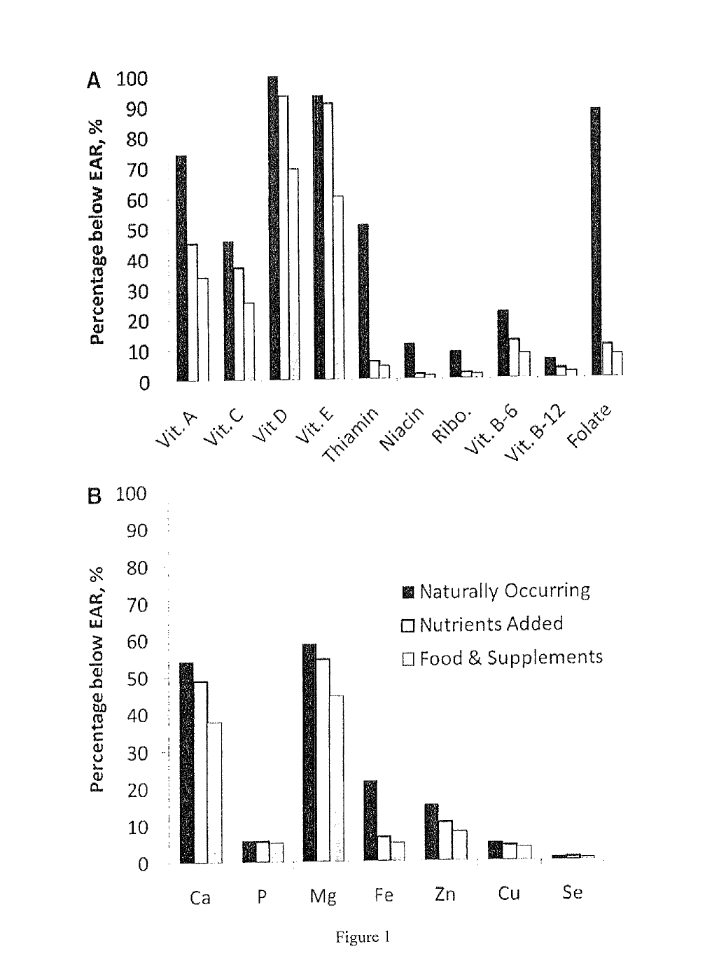

FIG. 2 includes two charts that illustrate the percentage of the population with usual intake below the EAR. Subgroups shown had the lowest (A, children aged 2 to 8 years) and highest (B, females aged 14 to 18 years) prevalence of inadequate intakes among the total population aged 2 to 18 years.

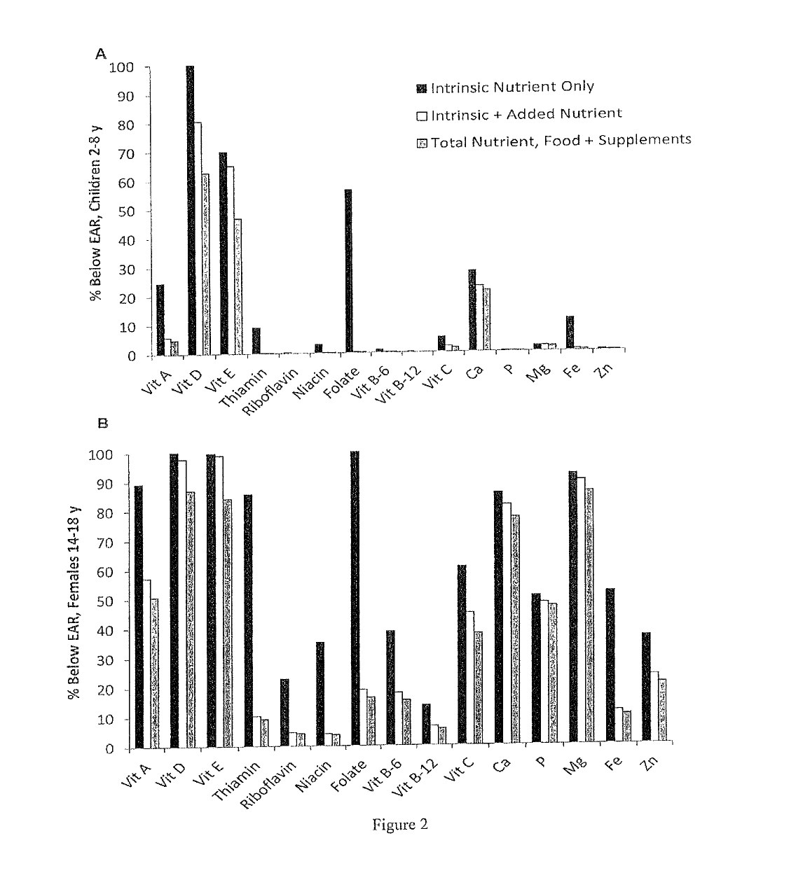

### DETAILED DESCRIPTION

This disclosure features food compositions that provide all of the essential nutrients to children and adults, in foods that most consider to taste good. The food compositions can be shelf-stable for up to two years. Each food composition is made with real foods. As needed in order to comprise all of the essential nutrients, the compositions can be enriched with vitamins and minerals. The compositions can be but need not be dehydrated. If they are dehydrated they can be re-constituted by adding hot water and stirring vigorously for several minutes.

Table 6 describes all of the essential nutrients that are included in the subject food compositions, giving the preferred amount (with preferred range given in parentheses), the minimum amount (lowest end), and a recommended maximum amount (highest end) per serving.

TABLE 6

| Nutrient | Preferred amount | | Lowest end | Highest end* | |
|---|---|---|---|---|---|
| Calories (kcal) | 350 | (200-500) | 1 | 1,000 | |
| Water (mL) | 300 | (250-350) | 10 | 500 | |
| Omega-3 fat (mg) | 1,500 | (1,000-3,000) | 1 | 5,000 | |
| Omega-6 fat (mg) | 1,500 | (1,000-3,000) | 1 | 5,000 | |
| Amino acids: a | To yield 25 grams | | 1 | 100 | |
| mixture of leucine, | (20-30). May be | | | | |
| isoleucine, lysine, | inherent in foods or | | | | |
| methionine, cysteine, | added as free amino | | | | |
| phenylalanine, | acids or peptides | | | | |
| tyrosine, tryptophan, | | | | | |
| threonine, valine, | | | | | |
| histidine, arginine | | | | | |
| Vitamin A (IU) | 2,500 | (1,000-3,500) | 1 | 3,000 | mcg |
| (5,000 IU = 1,500 mcg) | | | | | |
| Vitamin C (mg) | 30 | (15-45) | 1 | 2,000 | |
| Calcium (mg) | 500 | (250-750) | 1 | 2,500 | |
| Iron (mg) | 9 | | 1 | 45 | |
| Vitamin D (IU) | 200 | (100-400) | 1 | 100 | mcg |
| (400 IU = 10 mcg) | | | | | |
| Vitamin E (IU) | 15 | (10-30) | 1 | 1,000 | mg |
| (30 IU = 20 mg) | | | | | |

| | | | | |
|---|---|---|---|---|
| Vitamin K (mcg) | 40 | (10-80) | 3 | n/d |
| | | | | (no data) |
| Thiamin mg | 0.75 | (0.25-1.5) | 0.1 | n/d |
| Riboflavin mg | 0.85 | (0.4-1.7) | 0.1 | n/d |
| Niacin mg | 10 | (3-20) | 1 | 35 |
| Choline (mg) | 100 | (50-300) | 1 | 1,000 |
| Vitamin B6 mg | 1 | (0.5-2) | 0.1 | 100 |
| Folate mcg | 200 | (100-400) | 1 | 1,000 |
| Vitamin B12 mcg | 3 | (1-6) | 1 | n/d |
| Biotin mcg | 150 | (50-300) | 1 | n/d |
| Pantothenic acid mg | 5 | (1-10) | 1 | n/d |
| Phosphorus mg | 500 | (100-1,000) | 1 | 4,000 |
| Iodine mcg | 75 | (25-150) | 1 | 1,100 |
| Magnesium mg | 200 | (50-400) | 1 | 350 mg when added |
| | | | | as a supplement due |
| | | | | to increased chance |
| | | | | of gastrointestinal |
| | | | | disturbances. No |
| | | | | such problems |
| | | | | associated with |
| | | | | magnesium found in |
| | | | | foods. |
| Zinc mg | 7.5 | (1-15) | 0.5 | 40 |
| Selenium mcg | 35 | (10-70) | 1 | 400 |
| Copper mg | 1 | (0.25-2) | 0.1 | 10 |
| Manganese mg | 1 | (0.1-2) | 0.001 | 11 |
| Chromium mcg | 60 | (10-120) | 1 | n/d |
| Molybdenum mcg | 37.5 | (10-75) | 1 | 2,000 |
| Chloride mg | 1,700 | (500-3,400) | 1 | 3,600 |

*Not to exceed Upper limit of safety.

Table 7 includes estimated tolerable upper limit values. It is believed but not required that the subject food compositions should not provide more than this value. The Food and Nutrition Board of the Institute of Medicine has set the Dietary Reference Intakes (DRIs): Tolerable Upper Limit Intake Levels for the essential nutrients. The upper limit is the highest level of daily nutrient intake that is likely to pose no risk of adverse health effects to almost all individuals in the general population. These do not apply to someone under medical supervision. The total intake represents nutrients from food, water, and supplements. Upper limits could not be established for some nutrients due to lack of data, so staying close to 100% DV is advisable.

TABLE 7

| | |
|---|---|
| | Tolerable Upper Intake Levels |
| Nutrients with Percentage Daily | (based on males 19-30 years; |
| Value (% DV), and the 100% DV | the values vary slightly by |
| amount | age and gender) |

| Vitamin A 5,000 IU (1,500 mcg) | 3,000 | mcg |
|---|---|---|
| Vitamin C 60 mg | 2,000 | |
| Calcium 1,000 mg | 2,500 | |
| Iron 18 mg | 45 | |
| Vitamin D 400 IU (=10 mcg) | 100 | mcg |
| Vitamin E 30 IU (20 mg) | 1,000 | mg |

| | |
|---|---|
| Vitamin K 80 mcg | n/d |
| | (no data) |
| Thiamin 1.5 mg | n/d |
| Riboflavin 1.7 mg | n/d |
| Niacin 20 mg | 35 |
| Vitamin B6 2 mg | 100 |
| Folate 400 mcg | 1,000 |
| Vitamin B12 6 mcg | n/d |
| Biotin 300 mcg | n/d |
| Pantothenic acid 10 mg | n/d |
| Phosphorus 1,000 mg | 4,000 |
| Iodine 150 mcg | 1,100 |
| Magnesium 400 mg | 350 (This relates only to |
| | magnesium from supplements, |
| | due to increased chance of |
| | gastrointestinal disturbances. |
| | No such effects have been |
| | observed in foods.) |
| Zinc 15 mg | 40 |
| Selenium 70 mcg | 400 |
| Copper 2 mg | 10 |
| Manganese 2 mg | 11 |
| Chromium 120 mcg | n/d |
| Molybdenum 75 mcg | 2,000 |
| Chloride 3,400 mg | 3,600 |

### Sources of Nutrients and Essential Nutrients

The subject food compositions contain real foods that provide different macronutrients. Table 8 shows which nutrients are in existing real foods that can be used in the subject food compositions (preferred) and others that could alternatively be used.

TABLE 8

| Major | Foods used in | |
|---|---|---|
| macronutrient | existing products | Other foods |
| supplied | (preferred) | that could be used |
| Carbohydrate | Rice | |
| | Brown sugar, white sugar | Evaporated Cane Juice |
| | Turbinado Sugar | Mannose |
| | Oats | Galactose |
| | Apples | Sucrose |
| | Tomato | Juice-White Grape, Apple |
| | Fructose | Coconut Palm Sugar |
| | Lactose | Noodle/Pasta |
| | Butter milk | Agave Syrup |
| | Maltodextrin | Raisins |
| | Corn starch | |
| | Carrot | Gum Acacia |
| | Instant rice | Rice Crisps |
| | Red bell peppers | Sucanat |
| | Durum wheat flour | Quinoa |
| | Mushroom | Sorghum |
| | Potato | Teff |
| | Spinach | Amaranth |
| | Sweet corn | Cassava Flour |
| | Molasses | Corn Flour |
| | Beet | Maple Syrup |
| | | Honey |
| | | Bread Crumbs |
| Protein | Textured Vegetable Protein | Pea Protein |
| | Milk Protein Isolate | Egg |
| | Non-fat dry milk | |
| | Milk protein (concentrate) | |
| | Whole milk powder | |
| | Oat protein | |
| | Mozzarella cheese | |
| | Whey protein | |
| | Yeast extract | |
| | Red beans | |
| | Chicken | |
| | Parmesan cheese | |
| | Dehydrated cheddar cheese | |
| | Vermont cheddar cheese | |
| | (cultured milk, salt, | |
| | enzymes) | |
| | Blue cheese (cultured | |
| | pasteurized milk, salt, | |
| | enzymes) | |
| | Peanut flour | |
| Fat | Rice bran oil | Sunflower |
| | Butter (cream, salt) | Soybean |
| | Butter milk (dry) | Milk Fat |
| | Extra Virgin Olive oil | Palm Oil |
| | Butter oil | Coconut Fat |
| | Sweet cream solids | Coconut Oil |
| | Ghee | Peanut Oil |
| | Flax | Corn Oil |
| | High-oleic safflower oil | Hemp |
| | Sesame oil | Rape Seed Oil |
| Fiber | Hulled hemp seeds | Pectin |
| | Oat fiber | Psyllium |
| | Cellulose Gum | Gums |
| | Carrageenan | Lignin |

### Types of Flavorings/Seasonings in the Subject Food Compositions

The flavors of food compositions can be enhanced with flavorings, such as those set forth in Table 9 (which are preferred in some cases but not required).

TABLE 9

| | Ingredient used in | |
|---|---|---|
| | existing products | Other ingredients |
| Classification | (preferred) | that could be used |
| Seasoning | Sea salt | |
| | Natural flavor | |
| | Citric acid | Barbecue |
| | Vinegar powder | Cajun |
| | (maltodextrin, vinegar, | |
| | food starch) | |
| | Mustard | Cheese |
| | | Chicken & Herb |
| | | Butter & Herb |
| | Soy sauce (fermented | Thai |
| | soybeans, wheat, salt) | |
| | | Sour Cream & Onion |
| | | Chili Cheese |
| | | Jalepeno Cheese |
| | | Cheddar Cheese |
| | | Ranch |
| | | Asian Chili Garlic |
| | | Teriyaki Style |
| | | Roasted Red Pepper |
| Herbs/spice | Onion | Scallions |
| (plant) | Garlic | Thyme |
| | Oregano | Sage |
| | Red pepper (Cayenne) | Shallots |
| | Basil | Caraway |
| | Ground cinnamon | Fennel |
| | Black pepper | Cloves |
| | Lime | Ginger |
| | Cilantro | Curry |
| | Celery extract | Marjoram |
| | Paprika | Parsley |
| | Cumin | Poppy Seeds |
| | Chili powder | Rosemary |
| | Coriander | Sesame Seeds |
| | Nutmeg | Tarragon |
| | Ancho pepper | All Spice |
| | Chipotle pepper | Lavender |
| | Guajillo pepper | Saffron |
| | Tumeric | Mint Leaves |
| | Chives | Bay Leaves |
| | Dill | Anise |
| | Celery | Vanilla Bean |
| | Tamarind powder | Hibiscus Blossoms |
| | | Lemon Grass |
| | | Cardamon |
| | | Cayene |
| | | Chervil |
| | | Fenugreek |
| | | Garham Masala |
| Others | Soy Lecithin | |
| | Cellulose | Ascorbic |
| | Gum (xantham) | Furmaric |
| | Malic acid | Tartaric |
| | Egg yolk | Phosphoric |
| | Lactic acid | Fruit |
| | Hot sauce | Vegetable |
| | Mono- and di-glycerides | Herb |
| | Disodium inosinate | Bark |
| | Disodium guanylate | Bud |
| | | Root |
| | | Dairy |
| | Color added (Yellow 5 | Glycerin |
| | Lake, Yellow 6 Lake, | |
| | Yellow 5, Yellow 6) | |
| | Lactic acid | |
| | Fish powder | |

### Sources of Essential Nutrients in the Food Compositions

Some essential nutrients are naturally occurring in the foods that can be used in the subject food compositions, and others need to be added to meet the 50% DV goal, to make sure all of the essential nutrients are included, or both reasons. The amount of each essential nutrient added depends upon the foods used. Non-limiting, illustrative examples are given in table 10.

TABLE 10

| Major | Essential | |
|---|---|---|
| essential | nutrients used in | |
| nutrient | existing products | Other nutrient forms |
| category | (preferred) | that could be used |
| Vitamins | Vitamin A (Palmitate) | Beta-Carotene |
| | | Vitamin A Acetate |
| | Vitamin B1 (Thiamine | Thiamine Mononitrate, |
| | Mononitrate) | |
| | Vitamin B2 (Riboflavin) | Riboflavin |
| | Vitamin B3 (Niacinamide) | Niacinamide |
| | Vitamin B6 (Pyridoxine) | Pyridoxine |
| | | Hydrochloride |
| | Vitamin K | |
| | Vitamin B5 (Pantothenic | D-Calcium Pantothenate |
| | Acid) | |
| | Vitamin B12 | Cyanocobalamin |
| | (Cyanocobalamin) | |
| | Vitamin B8 (Biotin) | D-Biotin |
| | Vitamin C (Ascorbic Acid) | Ascorbic Acid |
| | Vitamin D (Cholecalciferol) | Cholecalciferol |
| | Vitamin E (Tocopheryl | dl-Alpha-Tocopheryl |
| | Acetate) | Acetate |
| | | dl-Alpha-Tocopheryl Acid |
| | | Succinate |
| | | Vitamin E Acetate |
| | Folic Acid | |
| Minerals/ | Manganese Sulfate | Manganese Glucinate |
| trace | Copper Oxide | Copper Gluconate |
| elements | | Copper Sulfate |
| | Chromium Chloride | Chromium Picolinate |
| | Sodium Molybdate | Molybdenum (Sodium |
| | | Molydate) |
| | Ferric Orthophosphate | Ferrous Fumarate |
| | Ferrous Sulfate | Carbonyl Iron |
| | Calcium Phosphate | Dibasic Calcium Phosphate |
| | Calcium caseinate | Calcium Carbonate |
| | | Calcium Citrate |
| | Potassium Iodide | |
| | Magnesium Oxide | Magnesium Stearate |
| | | Magnesium Aspartate |
| | | Magnesium Silicate |
| | Zinc Oxide | Zinc Picollinate |
| | Sodium Selenite | Selenium (Sodium |
| | | Selenate) |
| | | Sodium Ascorbate |
| | | Croscarmellose Sodium |
| | | Vanadium (Sodium |
| | | Metavanadate) |
| | Potassium Phosphate | |

Following are several illustrative, non-limiting examples of foods that are formulated under the present disclosure, and their nutrient analyses. These illustrate certain aspects of the invention. Many other foods can be formulated per this invention.

### Food 1: Apple Oatmeal

Apple Oatmeal Formulation

| Amount of Boiling Water: | ⅔ cup |
|---|---|
| Ingredients: | grams |
| Thick Rolled Oats (21.69 umol TE/g) | 20.00 |
| (where TE stands for Trolox | |
| Equivalents, per g dry matter) | |
| Milk protein isolate | 13.00 |
| Quick Oats (21.69 umol TE/g) | 12.00 |
| Turbinado Sugar | 12.00 |
| Soy Flake | 11.50 |
| Apple, ⅙″ Diced, Chewy, Air Dried | 8.00 |
| (150 umol/TE/g) | |
| High Oleic Safflower Oil | 4.00 |
| Flax Seed Powder | 3.80 |
| Coconut Oil | 3.00 |
| Dipotassium Phosphate | 0.260 |
| Oat Fiber | 1.00 |
| Dried Whole Egg Powder | 1.00 |
| Ground Cinnamon (1432 umol TE/g) | 0.30 |
| Bag Butter Powder | 0.30 |
| Dicalcium Phosphate | 0.940 |
| Sea Salt | 0.25 |
| Magnesium Oxide | 0.1450 |
| DL-Alpha Tocopheryl Acetate (Vitamin E) | 0.0300 |
| Ascorbic Acid (Vitamin C) | 0.0280 |
| Vitamin A Palmitate | 0.0100 |
| Niacinamide (Vitamin B3) | 0.0095 |
| Zinc Oxide | 0.0061 |
| D-Calcium Pantothenate (Vitamin B5) | 0.0049 |
| Ferric Orthophosphate | 0.0048 |
| Vitamin D3 | 0.00193 |
| Pyridoxine HCL (Vitamin B6) | 0.00096 |
| Riboflavin (Vitamin B2) | 0.00074 |
| Copper Oxide | 0.00071 |
| Thiamine Mononitrate (Vitamin B1) | 0.00058 |
| Manganese Sulfate | 0.00035 |
| Folic Acid | 0.00016 |
| D-Biotin (Vitamin B8) | 0.00015 |
| Potassium Iodide | 0.000075 |
| Chromium Chloride | 0.000049 |
| Vitamin K | 0.00004 |
| Sodium Molybdate | 0.0000145 |
| Selenium | 0.0120 |
| Cyanocobalain (vitamin B-12) | 0.00000165 |
| Total: | 91.606 |

Apple Oatmeal

| Nutrient Analysis | Yield: | 1 serving |
|---|---|---|
| | No. Ingredients: | 38 |

| Nutrient | Value | Unit | Goal | % of goal |
|---|---|---|---|---|
| Weight | 92.154 | g | | |
| Kilocalories | 352.127 | kcal | 2000.000 | 18% |
| Protein | 25.941 | g | 50.000 | 52% |
| Carbohydrate | 43.215 | g | 300.000 | 14% |
| Fat, Total | 11.797 | g | 65.000 | 18% |
| Alcohol | 0.000 | g | | |
| Cholesterol | 36.602 | mg | 300.000 | 12% |
| Saturated Fat | 3.741 | g | 20.000 | 19% |
| Monounsaturated Fat | 3.662 | g | | |
| Polyunsaturated Fat | 1.581 | g | | |
| MFA 18:1, Oleic | 3.705 | g | | |
| PFA 18:2, Linoleic | 0.738 | g | | |
| PFA 18:3, Linolenic | 0.760 | g | | |
| PFA 20:5, EPA | 0.000 | g | | |
| PFA 22:6, DHA | 0.000 | g | | |
| Trans Fatty Acid | 0.028 | g | | |
| Sodium | 148.279 | mg | 2400.000 | 6% |
| Potassium | 1739.310 | mg | 3500.000 | 50% |
| Vitamin A (RE) | 1.633 | RE | | |
| Vitamin A (IU) | 2491.919 | IU | 5000.000 | 50% |
| Vitamin A (RAE) | 0.045 | μg | | |
| Beta-Carotene | 0.336 | μg | | |
| Alpha-Carotene | 0.003 | μg | | |
| Lutein (+Zeaxanthin) | 0.666 | μg | | |
| Beta-Cryptoxanthin | 0.387 | μg | | |
| Lycopene | 0.045 | μg | | |
| Vitamin C | 29.879 | mg | 60.000 | 50% |
| Calcium | 496.285 | mg | 1000.000 | 50% |
| Iron | 8.960 | mg | 18.000 | 50% |
| Vitamin D (ug) | 0.000 | μg | 10.000 | 0% |
| Vitamin D (IU) | 200.750 | IU | 400.000 | 50% |
| Vitamin E (mg) | | | 20.000 | |
| Vitamin E (IU) | 15.071 | IU | 30.000 | 50% |
| Alpha-Tocopherol | 0.010 | mg | | |
| Thiamin | 0.755 | mg | 1.500 | 50% |
| Riboflavin | 0.850 | mg | 1.700 | 50% |
| Niacin | 10.012 | mg | 20.000 | 50% |
| Pyridoxine (Vitamin B6) | 0.993 | mg | 2.000 | 50% |
| Folate (Total) | 200.308 | μg | 400.000 | 50% |
| Folate (DFE) | 0.018 | μg | | |
| Cobalamin (Vitamin B12) | 2.984 | μg | 6.000 | 50% |
| Biotin | 150.000 | μg | 300.000 | 50% |
| Pantothenic Acid | 4.986 | mg | 10.000 | 50% |
| Vitamin K | 40.109 | μg | 80.000 | 50% |
| Phosphorus | 500.060 | mg | 1000.000 | 50% |
| Iodine | 75.000 | μg | 150.000 | 50% |
| Magnesium | 199.869 | mg | 400.000 | 50% |
| Zinc | 7.484 | mg | 15.000 | 50% |
| Copper | 0.996 | mg | 2.000 | 50% |
| Manganese | 0.992 | mg | 2.000 | 50% |
| Selenium | 35.125 | μg | 70.000 | 50% |
| Fluoride | | μg | | |
| Chromium | 0.061 | mg | 0.120 | 50% |
| Molybdenum | 37.500 | μg | 75.000 | 50% |
| Dietary Fiber, Total | 8.347 | g | 25.000 | 33% |
| Soluble Fiber | 1.889 | g | | |
| Insoluble Fiber | 4.664 | g | | |
| Crude Fiber | 0.073 | g | | |
| Sugar, Total | 17.326 | g | | |
| Glucose | 0.003 | g | | |
| Galactose | 0.000 | g | | |
| Fructose | 0.003 | g | | |
| Sucrose | 11.903 | g | | |
| Lactose | 0.000 | g | | |
| Maltose | 0.000 | g | | |
| Sugar Alcohol | 0.000 | g | | |
| Other Carbohydrate | 0.235 | g | | |
| Tryptophan | 301.687 | mg | | |
| Threonine | 957.068 | mg | | |
| Isoleucine | 1205.168 | mg | | |
| Leucine | 2035.369 | mg | | |
| Lysine | 1684.159 | mg | | |
| Methionine | 510.564 | mg | | |
| Cystine | 217.904 | mg | | |
| Phenylalanine | 1152.828 | mg | | |
| Tyrosine | 994.908 | mg | | |
| Valine | 1337.612 | mg | | |
| Arginine | 1570.888 | mg | | |
| Histidine | 611.491 | mg | | |
| Alanine | 856.008 | mg | | |
| Aspartic Acid | 2149.224 | mg | | |
| Glutamic Acid | 4601.709 | mg | | |
| Glycine | 704.795 | mg | | |
| Proline | 617.687 | mg | | |
| Serine | 1212.005 | mg | | |
| Moisture | 5.616 | g | | |
| Ash | 2.183 | g | | |
| Caffeine | 0.000 | mg | | |

DAILY VALUES/RDI - ADULT/CHILD

| Percentage of Kcals | |
|---|---|
| Protein | 27.1% |
| Carbohydrate | 45.2% |
| Fat, total | 27.7% |
| Alcohol | 0.0% |

### Food 2: Plain Oatmeal

Plain Oatmeal Formulation

| Amount of Boiling Water: | ⅔ cup |
|---|---|
| Ingredients: | grams |
| Thick Rolled Oats (21.69 umol TE/g) | 20.00 |
| Milk protein isolate | 13.00 |
| Quick Oats (21.69 umol TE/g) | 12.00 |
| Turbinado Sugar | 12.00 |
| Soy Flake | 11.50 |
| High Oleic Safflower Oil | 4.00 |
| Flax Seed Powder | 3.80 |
| Coconut Oil | 3.00 |
| Dipotassium Phosphate | 0.26 |
| Oat Fiber | 1.00 |
| Dried Whole Egg Powder | 1.00 |
| Nat. Vanilla Flavor | 0.40 |
| Bag Butter Powder | 0.30 |
| Ground Cinnamon | 0.30 |
| Dicalcium Phosphate | 0.94 |
| Sea Salt | 0.25 |
| Magnesium Oxide | 0.145 |
| DL-Alpha Tocopheryl Acetate (Vitamin E) | 0.0301 |
| Ascorbic Acid (Vitamin C) | 0.0295 |
| Vitamin A Palmitate | 0.01 |
| Niacinamide (Vitamin B3) | 0.0095 |
| Zinc Oxide | 0.0061 |
| D-Calcium Pantothenate (Vitamin B5) | 0.0049 |
| Ferric Orthophosphate | 0.0048 |
| Vitamin D3 | 0.00193 |
| Pyridoxine HCL (Vitamin B6) | 0.00098 |
| Riboflavin (Vitamin B2) | 0.00074 |
| Copper Oxide | 0.00073 |
| Thiamine Mononitrate (Vitamin B1) | 0.00058 |
| Manganese Sulfate | 0.00037 |
| Folic Acid | 0.00016 |
| D-Biotin (Vitamin B8) | 0.00015 |
| Potassium Iodide | 0.000075 |
| Chromium Chloride | 0.000049 |
| Vitamin K | 0.00004 |
| Sodium Molybdate | 0.0000145 |
| Selenium | 0.012000 |
| Cyanocobalain (vitamin B-12) | 0.00000165 |
| Total: | 84.008 |

Plain Oatmeal

| Nutrient Analysis | Yield: | 1 serving |
|---|---|---|
| | No. Ingredients: | 38 |

| Nutrient | Value | Unit | Goal | % of goal |
|---|---|---|---|---|
| Weight | 84.616 | g | | |
| Kilocalories | 331.559 | kcal | 2000.000 | 17% |
| Protein | 25.884 | g | 50.000 | 52% |
| Carbohydrate | 37.845 | g | 300.000 | 13% |
| Fat, Total | 11.677 | g | 65.000 | 18% |
| Alcohol | 0.000 | g | | |
| Cholesterol | 36.602 | mg | 300.000 | 12% |
| Saturated Fat | 3.721 | g | 20.000 | 19% |
| Monounsaturated Fat | 3.657 | g | | |
| Polyunsaturated Fat | 1.546 | g | | |
| MFA 18:1, Oleic | 3.705 | g | | |
| PFA 18:2, Linoleic | 0.738 | g | | |
| PFA 18:3, Linolenic | 0.760 | g | | |
| PFA 20:5, EPA | 0.000 | g | | |
| PFA 22:6, DHA | 0.000 | g | | |
| Trans Fatty Acid | 0.028 | g | | |
| Sodium | 148.283 | mg | 2400.000 | 6% |
| Potassium | 1745.680 | mg | 3500.000 | 50% |
| Vitamin A (RE) | 0.088 | RE | | |
| Vitamin A (IU) | 2524.995 | IU | 5000.000 | 50% |
| Vitamin A (RAE) | 0.045 | μg | | |
| Beta-Carotene | 0.336 | μg | | |
| Alpha-Carotene | 0.003 | μg | | |
| Lutein (+Zeaxanthin] | 0.666 | μg | | |
| Beta-Cryptoxanthin | 0.387 | μg | | |
| Lycopene | 0.045 | ug | | |
| Vitamin C | 29.834 | mg | 60.000 | 50% |
| Calcium | 504.743 | mg | 1000.000 | 50% |
| Iron | 8.936 | mg | 18.000 | 50% |
| Vitamin D (ug) | 0.000 | μg | 10.000 | 0% |
| Vitamin D (IU) | 200.750 | IU | 400.000 | 50% |
| Vitamin E (mg) | | | 20.000 | |
| Vitamin E (IU) | 15.121 | IU | 30.000 | 50% |
| Alpha-Tocopherol | 0.010 | mg | | |
| Thiamin | 0.748 | mg | 1.500 | 50% |
| Riboflavin | 0.846 | mg | 1.700 | 50% |
| Niacin | 9.977 | mg | 20.000 | 50% |
| Pyridoxine (Vitamin B6) | 0.985 | mg | 2.000 | 49% |
| Folate (Total) | 200.308 | μg | 400.000 | 50% |
| Folate (DFE) | 0.018 | μg | | |
| Cobalamin (Vitamin B12) | 2.984 | μg | 6.000 | 50% |
| Biotin | 150.000 | μg | 300.000 | 50% |
| Pantothenic Acid | 4.963 | mg | 10.000 | 50% |
| Vitamin K | 40.109 | μg | 80.000 | 50% |
| Phosphorus | 497.358 | mg | 1000.000 | 50% |
| Iodine | 75.000 | μg | 150.000 | 50% |
| Magnesium | 198.711 | mg | 400.000 | 50% |
| Zinc | 7.469 | mg | 15.000 | 50% |
| Copper | 1.004 | mg | 2.000 | 50% |
| Manganese | 1.003 | mg | 2.000 | 50% |
| Selenium | 35.009 | μg | 70.000 | 50% |
| Fluoride | | μg | | |
| Chromium | 0.061 | mg | 0.120 | 50% |
| Molybdenum | 37.500 | μg | 75.000 | 50% |
| Dietary Fiber, Total | 7.613 | g | 25.000 | 30% |
| Soluble Fiber | 1.889 | g | | |
| Insoluble Fiber | 4.664 | g | | |
| Crude Fiber | 0.073 | g | | |
| Sugar, Total | 12.858 | g | | |
| Glucose | 0.003 | g | | |
| Galactose | 0.000 | g | | |
| Fructose | 0.003 | g | | |
| Sucrose | 11.903 | g | | |
| Lactose | 0.000 | g | | |
| Maltose | 0.000 | g | | |
| Sugar Alcohol | 0.000 | g | | |
| Other Carbohydrate | 0.000 | g | | |
| Tryptophan | 301.687 | mg | | |
| Threonine | 957.068 | mg | | |
| Isoleucine | 1205.168 | mg | | |
| Leucine | 2035.369 | mg | | |
| Lysine | 1684.159 | mg | | |
| Methionine | 510.564 | mg | | |
| Cystine | 217.904 | mg | | |
| Phenylalanine | 1152.828 | mg | | |
| Tyrosine | 994.908 | mg | | |
| Valine | 1337.612 | mg | | |
| Arginine | 1570.888 | mg | | |
| Histidine | 611.491 | mg | | |
| Alanine | 856.008 | mg | | |
| Aspartic Acid | 2149.224 | mg | | |
| Glutamic Acid | 4601.709 | mg | | |
| Glycine | 704.795 | mg | | |
| Proline | 617.687 | mg | | |
| Serine | 1212.005 | mg | | |
| Moisture | 5.656 | g | | |
| Ash | 2.183 | g | | |
| Caffeine | 0.000 | mg | | |

DAILY VALUES/RDI - ADULT/CHILD

| Percentage of Kcals | |
|---|---|
| Protein | 28.8% |
| Carbohydrate | 42.0% |
| Fat, total | 29.2% |
| Alcohol | 0.0% |

### Food 3: Breakfast Rice

Breakfast Rice Formulation

| Amount of Boiling Water: | ⅔ cup |
|---|---|
| Ingredients: | grams |
| Instant White Rice Enriched | 28.00 |
| Milk protein isolate | 14.70 |
| Turbinado sugar | 12.00 |
| Soy Flake | 12.00 |
| High Oleic Safflower Oil | 4.00 |
| Flax Seed Powder | 3.80 |
| Oat Fiber0 | 3.50 |
| Coconut Oil | 3.00 |
| Dipotassium Phosphate | 0.30 |
| Dried Whole Egg Powder | 1.00 |
| Nat. Vanilla Flavor | 0.40 |
| Bag Butter Powder | 0.30 |
| Sea Salt | 0.25 |
| Dicalcium Phosphate | 0.48 |
| Magnesium Oxide | 0.135 |
| Ground Cinnamon (1432 umol/TE/g) | 0.10 |
| DL-Alpha Tocopheryl Acetate (Vitamin E) | 0.03 |
| Ascorbic Acid (Vitamin C) | 0.0295 |
| Vitamin A Palmitate | 0.01 |
| Niacinamide (Vitamin B3) | 0.0085 |
| Zinc Oxide | 0.0057 |
| The Wright Group, Ferric Orthophosphate | 0.0050 |
| D-Calcium Pantothenate (Vitamin B5) | 0.0049 |
| Vitamin D3 | 0.0019 |
| Pyridoxine HCL (Vitamin B6) | 0.00096 |
| Riboflavin (Vitamin B2) | 0.00079 |
| Copper Oxide | 0.00072 |
| Thiamine Mononitrate (Vitamin B1) | 0.0006 |
| D-Biotin (Vitamin B8) | 0.00015 |
| Folic Acid | 0.000114 |
| Potassium Iodide | 0.000075 |
| Manganese Sulfate | 0.000380 |
| Chromium Chloride | 0.000048 |
| Vitamin K | 0.00004 |
| Sodium Molybdate | 0.0000135 |
| Selenium | 0.011 |
| Cyanocobalain (vitamin B-12) | 0.00000152 |
| Total: | 84.075 |

Breakfast Rice

| Nutrient Analysis | Yield: | 1 serving |
|---|---|---|
| | No. Ingredients: | 38 |

| Nutrient | Value | Unit | Goal | % of goal |
|---|---|---|---|---|
| Weight | 85.174 | g | | |
| Kilocalories | 332.699 | kcal | 2000.000 | 17% |
| Protein | 24.961 | g | 50.000 | 50% |
| Carbohydrate | 42.377 | g | 300.000 | 14% |
| Fat, Total | 9.665 | g | 65.000 | 15% |
| Alcohol | 0.000 | g | | |
| Cholesterol | 39.186 | mg | 300.000 | 13% |
| Saturated Fat | 3.307 | g | 20.000 | 17% |
| Monounsaturated Fat | 3.716 | g | | |
| Polyunsaturated Fat | 0.636 | g | | |
| MFA 18:1, Oleic | 3.704 | g | | |
| PFA 18:2, Linoleic | 0.738 | g | | |
| PFA 18:3, Linolenic | 0.760 | g | | |
| PFA 20:5, EPA | 0.000 | g | | |
| PFA 22:6, DHA | 0.000 | g | | |
| Trans Fatty Acid | 0.030 | g | | |
| Sodium | 185.133 | mg | 2400.000 | 8% |
| Potassium | 1769.772 | mg | 3500.000 | 51% |
| Vitamin A (RE) | 0.029 | RE | | |
| Vitamin A (IU) | 2524.229 | IU | 5000.000 | 50% |
| Vitamin A (RAE) | 0.015 | μg | | |
| Beta-Carotene | 0.112 | μg | | |
| Alpha-Carotene | 0.001 | μg | | |
| Lutein (+Zeaxanthin) | 0.222 | μg | | |
| Beta-Cryptoxanthin | 0.129 | μg | | |
| Lycopene | 0.015 | μg | | |
| Vitamin C | 29.854 | mg | 60.000 | 50% |
| Calcium | 502.041 | mg | 1000.000 | 50% |
| Iron | 9.005 | mg | 18.000 | 50% |
| Vitamin D (ug) | 0.000 | μg | 10.000 | 0% |
| Vitamin D (IU) | 198.600 | IU | 400.000 | 50% |
| Vitamin E (mg) | | | 20.000 | |
| Vitamin E (IU) | 15.077 | IU | 30.000 | 50% |
| Alpha-Tocopherol | 0.036 | mg | | |
| Thiamin | 0.751 | mg | 1.500 | 50% |
| Riboflavin | 0.847 | mg | 1.700 | 50% |
| Niacin | 9.986 | mg | 20.000 | 50% |
| Pyridoxine (Vitamin B6) | 1.010 | mg | 2.000 | 50% |
| Folate (Total) | 201.434 | μg | 400.000 | 50% |
| Folate (DFE) | 2.235 | μg | | |
| Cobalamin (Vitamin B12) | 3.024 | μg | 6.000 | 50% |
| Biotin | 150.840 | μg | 300.000 | 50% |
| Pantothenic Acid | 4.963 | mg | 10.000 | 50% |
| Vitamin K | 40.074 | μg | 80.000 | 50% |
| Phosphorus | 497.745 | mg | 1000.000 | 50% |
| Iodine | 75.000 | μg | 150.000 | 50% |
| Magnesium | 200.273 | mg | 400.000 | 50% |
| Zinc | 7.488 | mg | 15.000 | 50% |
| Copper | 0.995 | mg | 2.000 | 50% |
| Manganese | 1.004 | mg | 2.000 | 50% |
| Selenium | 35.003 | μg | 70.000 | 50% |
| Fluoride | | μg | | |
| Chromium | 0.060 | mg | 0.120 | 50% |
| Molybdenum | 37.500 | μg | 75.000 | 50% |
| Dietary Fiber, Total | 7.096 | g | 25.000 | 28% |
| Soluble Fiber | 0.362 | g | | |
| Insoluble Fiber | 5.674 | g | | |
| Crude Fiber | 0.024 | g | | |
| Sugar, Total | 12.945 | g | | |
| Glucose | 0.001 | g | | |
| Galactose | 0.000 | g | | |
| Fructose | 0.001 | g | | |
| Sucrose | 11.903 | g | | |
| Lactose | 0.000 | g | | |
| Maltose | 0.000 | g | | |
| Sugar Alcohol | 0.000 | g | | |
| Other Carbohydrate | 21.977 | g | | |
| Tryptophan | 350.355 | mg | | |
| Threonine | 1111.953 | mg | | |
| Isoleucine | 1398.833 | mg | | |
| Leucine | 2383.369 | mg | | |
| Lysine | 1906.970 | mg | | |
| Methionine | 609.728 | mg | | |
| Cystine | 273.802 | mg | | |
| Phenylalanine | 1357.114 | mg | | |
| Tyrosine | 1155.149 | mg | | |
| Valine | 1579.270 | mg | | |
| Arginine | 1864.797 | mg | | |
| Histidine | 711.093 | mg | | |
| Alanine | 921.828 | mg | | |
| Aspartic Acid | 2497.790 | mg | | |
| Glutamic Acid | 5400.890 | mg | | |
| Glycine | 841.797 | mg | | |
| Proline | 735.249 | mg | | |
| Serine | 1417.470 | mg | | |
| Moisture | 1.908 | g | | |
| Ash | 1.774 | g | | |
| Caffeine | 0.000 | mg | | |

DAILY VALUES/RDI - ADULT/CHILD

| Percentage of Kcals | |
|---|---|
| Protein | 28.0% |
| Carbohydrate | 47.6% |
| Fat, total | 24.4% |
| Alcohol | 0.0% |

### Food 4: Granola

Granola Formulation

| Ingredients: | grams |
|---|---|
| Thick Rolled Oats | 30.00 |
| Milk protein isolate | 13.00 |
| Soy Flake | 11.50 |
| Flax Seed Powder, 30 mesh | 3.80 |
| Potassium Phosphate | 1.290 |
| Oat Fiber | 1.00 |
| Dried Whole Egg Powder | 1.00 |
| Ground Cinnamon | 0.45 |
| Nat. Vanilla Flavor | 0.40 |
| Bag Butter Powder | 0.30 |
| Calcium Phosphate | 0.2800 |
| Sea Salt | 0.25 |
| Magnesium Oxide | 0.155 |
| DL-Alpha Tocopheryl Acetate (Vitamin E) | 0.0301 |
| Ascorbic Acid (Vitamin C) | 0.0300 |
| Vitamin A Palmitate | 0.0100 |
| Niacinamide (Vitamin B3) | 0.01 |
| Zinc Oxide | 0.0063 |
| Ferric Orthophosphate | 0.0050 |
| D-Calcium Pantothenate (Vitamin B5) | 0.005 |
| Vitamin D3 | 0.00193 |
| Pyridoxine HCL (Vitamin B6) | 0.001 |
| Copper Oxide | 0.00075 |
| Riboflavin (Vitamin B2) | 0.0007 |
| Thiamine Mononitrate (Vitamin B1) | 0.0006 |
| Manganese Sulfate | 0.0004 |
| Folic Acid | 0.00016 |
| D-Biotin (Vitamin B8) | 0.00015 |
| Potassium Iodide | 0.000075 |
| Chromium Chloride | 0.000049 |
| Vitamin K | 0.00004 |
| Sodium Molybdate | 0.0000145 |
| Sodium Selenite | 0.000012 |
| Cyanocobalain (vitamin B-12) | 0.00000165 |
| Total: | 63.53 |
| Mix the following ingredients, | |
| then slowly add under agitation | |
| to the dry blend above. | |
| Water | 30.00 |
| Turbinado Sugar | 15.00 |
| Glycerin | 13.00 |
| Coconut Oil | 7.00 |
| Total: | 65.00 |
| Complete Batch Total: | 128.53 |

Granola

| Nutrient Analysis | Yield: | 1 serving |
|---|---|---|
| | No. Ingredients: | 37 |

| Nutrient | Value | Unit | Goal | % of goal |
|---|---|---|---|---|
| Weight | 98.527 | g | | |
| Kilocalories | 392.379 | kcal | 2000.000 | 20% |
| Protein | 25.590 | g | 50.000 | 51% |
| Carbohydrate | 52.635 | g | 300.000 | 18% |
| Fat, Total | 11.539 | g | 65.000 | 18% |
| Alcohol | 0.000 | g | | |
| Cholesterol | 36.602 | mg | 300.000 | 12% |
| Saturated Fat | 6.891 | g | 20.000 | 34% |
| Monounsaturated Fat | 0.625 | g | | |
| Polyunsaturated Fat | 1.102 | g | | |
| MFA 18:1, Oleic | 0.673 | g | | |
| PFA 18:2, Linoleic | 0.354 | g | | |
| PFA 18:3, Linolenic | 0.760 | g | | |
| PFA 20:5, EPA | 0.000 | g | | |
| PFA 22:6, DHA | 0.000 | g | | |
| Trans Fatty Acid | 0.028 | g | | |
| Sodium | 148.288 | mg | 2400.000 | 6% |
| Potassium | 1749.396 | mg | 3500.000 | 50% |
| Vitamin A (RE) | 0.133 | RE | | |
| Vitamin A (IU) | 2525.337 | IU | 5000.000 | 51% |
| Vitamin A (RAE) | 0.068 | μg | | |
| Beta-Carotene | 0.504 | μg | | |
| Alpha-Carotene | 0.005 | μg | | |
| Lutein (+Zeaxanthin) | 0.999 | μg | | |
| Beta-Cryptoxanthin | 0.580 | μg | | |
| Lycopene | 0.068 | μg | | |
| Vitamin C | 30.339 | mg | 60.000 | 51% |
| Calcium | 505.706 | mg | 1000.000 | 51% |
| Iron | 9.071 | mg | 18.000 | 50% |
| Vitamin D (ug) | 0.000 | μg | 10.000 | 0% |
| Vitamin D (IU) | 200.750 | IU | 400.000 | 50% |
| Vitamin E (mg) | | | 20.000 | |
| Vitamin E (IU) | 15.121 | IU | 30.000 | 50% |
| Alpha-Tocopherol | 0.017 | mg | | |
| Thiamin | 0.738 | mg | 1.500 | 49% |
| Riboflavin | 0.842 | mg | 1.700 | 50% |
| Niacin | 10.453 | mg | 20.000 | 52% |
| Pyridoxine (Vitamin B6) | 1.006 | mg | 2.000 | 50% |
| Folate (Total) | 200.317 | μg | 400.000 | 50% |
| Folate (DFE) | 0.027 | μg | | |
| Cobalamin (Vitamin B12) | 2.984 | μg | 6.000 | 50% |
| Biotin | 150.000 | μg | 300.000 | 50% |
| Pantothenic Acid | 5.064 | mg | 10.000 | 51% |
| Vitamin K | 40.175 | μg | 80.000 | 50% |
| Phosphorus | 237.484 | mg | 1000.000 | 24% |
| Iodine | 75.000 | μg | 150.000 | 50% |
| Magnesium | 208.861 | mg | 400.000 | 52% |
| Zinc | 7.672 | mg | 15.000 | 51% |
| Copper | 1.024 | mg | 2.000 | 51% |
| Manganese | 1.041 | mg | 2.000 | 52% |
| Selenium | 35.014 | μg | 70.000 | 50% |
| Fluoride | | μg | | |
| Chromium | 0.061 | mg | 0.120 | 50% |
| Molybdenum | 37.500 | μg | 75.000 | 50% |
| Dietary Fiber, Total | 7.493 | g | 25.000 | 30% |
| Soluble Fiber | 1.873 | g | | |
| Insoluble Fiber | 4.560 | g | | |
| Crude Fiber | 0.110 | g | | |
| Sugar, Total | 15.837 | g | | |
| Glucose | 0.005 | g | | |
| Galactose | 0.000 | g | | |
| Fructose | 0.005 | g | | |
| Sucrose | 14.879 | g | | |
| Lactose | 0.000 | g | | |
| Maltose | 0.000 | g | | |
| Sugar Alcohol | 0.000 | g | | |
| Other Carbohydrate | 0.000 | g | | |
| Tryptophan | 301.761 | mg | | |
| Threonine | 957.272 | mg | | |
| Isoleucine | 1205.387 | mg | | |
| Leucine | 2035.749 | mg | | |
| Lysine | 1684.523 | mg | | |
| Methionine | 510.681 | mg | | |
| Cystine | 217.991 | mg | | |
| Phenylalanine | 1153.047 | mg | | |
| Tyrosine | 995.112 | mg | | |
| Valine | 1337.948 | mg | | |
| Arginine | 1571.137 | mg | | |
| Histidine | 611.666 | mg | | |
| Alanine | 856.257 | mg | | |
| Aspartic Acid | 2149.881 | mg | | |
| Glutamic Acid | 4602.265 | mg | | |
| Glycine | 705.088 | mg | | |
| Proline | 618.316 | mg | | |
| Serine | 1212.297 | mg | | |
| Moisture | 5.498 | g | | |
| Ash | 2.158 | g | | |
| Caffeine | 0.000 | mg | | |

DAILY VALUES/RDI - ADULT/CHILD

| Percentage of Kcals | |
|---|---|
| Protein | 24.6% |
| Carbohydrate | 50.5% |
| Fat, total | 24.9% |
| Alcohol | 0.0% |

### Food 5: Pad Thai

Pad Thai Formulation

| Ingredients: | grams |
|---|---|
| Mung Bean Noodles | 25.00 |
| Soy flakes | 10.00 |
| Air Dried Carrot (4.36 umol TE/g) | 3.00 |
| Air Dried Green Onion (42.89 umol TE/g) | 1.00 |
| Pad Thai Sauce | 5.00 |
| High Oleic Safflower Oil (204 umol TE/g) | 5.00 |
| Reduced Sodium Soy Sauce Powder | 1.00 |
| Dark Brown Sugar | 10.00 |
| Nat Flavor Enhancer | 0.26 |
| Anchovy powder | 0.90 |
| Fish Sauce Powder | 0.50 |
| Partially Defatted Peanut Flour, 28% | 12.00 |
| fat dark roast | |
| Milk Protein Isolate | 6.00 |
| Flax Seed Powder, 14 mesh | 4.00 |
| Chicken Broth Powder | 7.00 |
| Deoiled Lecithin | 0.50 |
| Coconut Flavor Powder | 0.30 |
| Rice Vinegar Powder | 0.30 |
| Freeze Dried, Garlic Clove | 0.40 |
| Freeze Dried, Ginger Root | 0.20 |
| Air Dried Lime Peel Powder | 0.30 |
| Mineral, Dicalcium Phosphate | 0.94 |
| Mineral, Chromium Chloride | 0.000050 |
| Mineral, Copper Oxide | 0.00073 |
| Mineral, Ferric Orthophosphate | 0.0060 |
| Mineral, Magnesium Oxide | 0.145 |
| Mineral, Manganese Sulfate | 0.00044 |
| Mineral, Potassium Iodide | 0.000075 |
| Mineral, Dipotassium Phosphate | 0.34 |
| Mineral, Sodium Molybdate | 0.0000175 |
| Mineral, Selenium yeast (1 mg/gm) | 0.015 |
| Mineral, Zinc Oxide | 0.0063 |
| Vitamin B1, Thiamine Mononitrate | 0.00058 |
| Vitamin B12, Cyanocobalain | 0.0000024 |
| Vitamin B2, Riboflavin | 0.0008 |
| Vitamin B3, Niacinamide | 0.0096 |
| Vitamin B5, D-Calcium Pantothenate | 0.0049 |
| Vitamin B6, Pyridoxine HCL | 0.00099 |
| Vitamin B8, D-Biotin | 0.00015 |
| Vitamin B9, Folic Acid | 0.00015 |
| Vitamin C, Ascorbic Acid | 0.0295 |
| Vitamin D3 | 0.00205 |
| Vitamin E, DL-Alpha Tocopheryl Acetate | 0.0301 |
| Vitamin K | 0.00004 |
| Total weight in grams: | 94.19 |

### Food 6: Miso Soup

Miso Soup Formulation

| Boiling Water | 1 Cup |
|---|---|
| Ingredients: | grams |
| Freeze Dried Cod Powder | 10.00 |
| Red Miso Powder | 4.00 |
| Flavor Enhancer, 7103 wheat free | 0.50 |
| Dried Seaweed | 1.00 |
| Dried Whole Egg Powder Type W1 | 1.00 |
| Deoiled Lecithin | 0.50 |
| VegOtein P80 | 7.00 |
| Chicken Broth | 12.50 |
| Dipotassium Phosphate | 0.58 |
| Dicalcium Phosphate | 1.12 |
| Magnesium Oxide | 0.15 |
| DL-Alpha Tocopheryl Acetate (Vitamin E) | 0.03 |
| Ascorbic Acid (Vitamin C) | 0.0295 |
| Vitamin A Palmitate | 0.010 |
| Niacinamide (Vitamin B3) | 0.0088 |
| Zinc Oxide | 0.0073 |
| D-Calcium Pantothenate (Vitamin B5) | 0.0049 |
| Ferric Orthophosphate | 0.0075 |
| Vitamin D3 | 0.00197 |
| Pyridoxine HCL (Vitamin B6) | 0.00096 |
| Riboflavin (Vitamin B2) | 0.00077 |
| Copper Oxide | 0.00097 |
| Thiamine Mononitrate (Vitamin B1) | 0.00070 |
| Manganese Sulfate | 0.00100 |
| Folic Acid | 0.000196 |
| D-Biotin (Vitamin B8) | 0.00015 |
| Potassium Iodide | 0.000060 |
| Chromium Chloride | 0.000060 |
| Vitamin K | 0.00004 |
| Sodium Molybdate | 0.0000375 |
| Selenium | 0.035 |
| Cyanocobalain (vitamin B-12) | 0.00000295 |
| Total: | 38.48 |

Miso Soup

| Nutrient Analysis | Yield: | 1 serving |
|---|---|---|
| | No. Ingredients: | 32 |

| Nutrient | Value | Unit | Goal | % of goal |
|---|---|---|---|---|
| Weight | 38.485 | g | | |
| Kilocalories | 154.360 | kcal | 2000.000 | 8% |
| Protein | 25.184 | g | 50.000 | 50% |
| Carbohydrate | 3.282 | g | 300.000 | 1% |
| Fat, Total | 4.426 | g | 65.000 | 7% |
| Alcohol | 0.000 | g | | |
| Cholesterol | 97.290 | mg | 300.000 | 32% |
| Saturated Fat | 1.169 | g | 20.000 | 6% |
| Monounsaturated Fat | 0.332 | g | | |
| Polyunsaturated Fat | 0.605 | g | | |
| MFA 18:1, Oleic | 0.000 | g | | |
| PFA 18:2, Linoleic | 0.000 | g | | |
| PFA 18:3, Linolenic | 0.000 | g | | |
| PFA 20:5, EPA | 0.000 | g | | |
| PFA 22:6, DHA | 0.000 | g | | |
| Trans Fatty Acid | 0.024 | g | | |
| Sodium | 424.416 | mg | 2400.000 | 18% |
| Potassium | 538.494 | mg | 3500.000 | 15% |
| Vitamin A (RE) | 4.004 | RE | | |
| Vitamin A (IU) | 2521.722 | IU | 5000.000 | 50% |
| Vitamin A (RAE) | | | | |
| Beta-Carotene | | | | |
| Alpha-Carotene | | | | |
| Lutein (+Zeaxanthin) | | | | |
| Beta-Cryptoxanthin | | | | |
| Lycopene | | | | |
| Vitamin C | 30.022 | mg | 60.000 | 50% |
| Calcium | 500.370 | mg | 1000.000 | 50% |
| Iron | 9.037 | mg | 18.000 | 50% |
| Vitamin D [ug] | 0.000 | μg | 10.000 | 0% |
| Vitamin D (IU) | 198.250 | IU | 400.000 | 50% |
| Vitamin E (mg) | | | 20.000 | |
| Vitamin E (IU) | 15.122 | IU | 30.000 | 50% |
| Alpha-Tocopherol | | | | |
| Thiamin | 0.759 | mg | 1.500 | 51% |
| Riboflavin | 0.847 | mg | 1.700 | 50% |
| Niacin | 10.040 | mg | 20.000 | 50% |
| Pyridoxine (Vitamin B6) | 1.018 | mg | 2.000 | 51% |
| Folate (Total) | 199.356 | μg | 400.000 | 50% |
| Folate (DFE) | 0.000 | μg | | |
| Cobalamin (Vitamin B12) | 2.984 | μg | 6.000 | 50% |
| Biotin | 150.035 | μg | 300.000 | 50% |
| Pantothenic Acid | 5.049 | mg | 10.000 | 50% |
| Vitamin K | 40.000 | μg | 80.000 | 50% |
| Phosphorus | 502.059 | mg | 1000.000 | 50% |
| Iodine | 75.000 | μg | 150.000 | 50% |
| Magnesium | 200.407 | mg | 400.000 | 50% |
| Zinc | 7.557 | mg | 15.000 | 50% |
| Copper | 1.001 | mg | 2.000 | 50% |
| Manganese | 1.001 | mg | 2.000 | 50% |
| Selenium | 35.000 | μg | 70.000 | 50% |
| Fluoride | | μg | | |
| Chromium | 0.060 | mg | 0.120 | 50% |
| Molybdenum | 37.500 | μg | 75.000 | 50% |
| Dietary Fiber, Total | 0.839 | g | 25.000 | 3% |
| Soluble Fiber | 0.003 | g | | |
| Insoluble Fiber | 0.008 | g | | |
| Crude Fiber | | g | | |
| Sugar, Total | 0.021 | g | | |
| Glucose | 0.000 | g | | |
| Galactose | 0.000 | g | | |
| Fructose | 0.000 | g | | |
| Sucrose | 0.000 | g | | |
| Lactose | 0.000 | g | | |
| Maltose | 0.000 | g | | |
| Sugar Alcohol | 0.000 | g | | |
| Other Carbohydrate | | g | | |
| Tryptophan | 7.802 | mg | | |
| Threonine | 21.440 | mg | | |
| Isoleucine | 24.509 | mg | | |
| Leucine | 41.783 | mg | | |
| Lysine | 33.616 | mg | | |
| Methionine | 15.033 | mg | | |
| Cystine | 10.772 | mg | | |
| Phenylalanine | 25.478 | mg | | |
| Tyrosine | 19.937 | mg | | |
| Valine | 30.111 | mg | | |
| Arginine | 30.909 | mg | | |
| Histidine | 12.104 | mg | | |
| Alanine | 27.242 | mg | | |
| Aspartic Acid | 50.550 | mg | | |
| Glutamic Acid | 64.332 | mg | | |
| Glycine | 16.339 | mg | | |
| Proline | 18.437 | mg | | |
| Serine | 37.980 | mg | | |
| Moisture | 0.930 | g | | |
| Ash | 2.727 | g | | |
| Caffeine | 0.000 | mg | | |

DAILY VALUES/RDI - ADULT/CHILD

| Percentage of Kcals | |
|---|---|
| Protein | 65.5% |
| Carbohydrate | 8.5% |
| Fat, total | 25.9% |
| Alcohol | 0.0% |

### Food 7: Red Beans and Rice

Red Bean & Rice

| Size: | |
|---|---|
| Ingredients: | grams |
| Minute Rice | 10.50 |
| Drum Dried Tomato Paste Flakes (6.94 umol TE/g) | 0.66 |
| Drum Dried Red Bean Powder (86.06 umol TE/g) | 20.00 |
| Mirepoix Powder | 2.50 |
| Freeze Dried Raw Garlic | 0.50 |
| Freeze Dried Oregano | 0.10 |
| Soy Flakes | 12.00 |
| Sea Salt | 0.70 |
| Ground Cumin | 0.20 |
| Tomato Flavor | 0.20 |
| Milk Protein Isolate | 12.50 |
| High Oleic Safflower Oil | 5.00 |
| Lecithin | 0.40 |
| Chili Seasoning | 2.00 |
| Flavor Enhancer, Wheat Free | 0.40 |
| Freeze Dried Cilantro | 0.30 |
| 14 mesh Flax Seed | 3.80 |
| Crystalline Fructose | 1.00 |
| Sharp Vermont Cheddar Seasoning | 4.00 |
| Dipotassium Phosphate | 1.20 |
| Dicalcium Phosphate | 0.95 |
| Magnesium Oxide | 0.121 |
| DL-Alpha Tocopheryl Acetate (Vitamin E) | 0.03 |
| Ascorbic Acid (Vitamin C) | 0.0245 |
| Vitamin A Palmitate | 0.082 |
| Niacinamide (Vitamin B3) | 0.0090 |
| Zinc Oxide | 0.0058 |
| D-Calcium Pantothenate (Vitamin B5) | 0.0049 |
| Ferric Orthophosphate | 0.0048 |
| Vitamin D3 | 0.00193 |
| Pyridoxine HCL (Vitamin B6) | 0.00090 |
| Riboflavin (Vitamin B2) | 0.00077 |
| Copper Oxide | 0.00061 |
| Thiamine Mononitrate (Vitamin B1) | 0.00062 |
| Folic Acid | 0.00010 |
| D-Biotin (Vitamin B8) | 0.00015 |
| Potassium Iodide | 0.000075 |
| Chromium Chloride | 0.000049 |
| Vitamin K | 0.000033 |
| Sodium Molybdate | 0.0000145 |
| Selenium | 0.008 |
| Cyanocobalain (vitamin B-12) | 0.00000180 |
| Total: | 79.2053 |

Red Beans & Rice

| Nutrient Analysis | Yield: | 1 serving |
|---|---|---|
| | No. Ingredients: | 42 |

| Nutrient | Value | Unit | Goal | % of goal |
|---|---|---|---|---|
| Weight | 79.205 | g | | |
| Kilocalories | 304.776 | kcal | 2000.000 | 15% |
| Protein | 26.286 | g | 50.000 | 53% |
| Carbohydrate | 33.015 | g | 300.000 | 11% |
| Fat, Total | 9.042 | g | 65.000 | 14% |
| Alcohol | 0.000 | g | | |
| Cholesterol | 23.158 | mg | 300.000 | 8% |
| Saturated Fat | 1.441 | g | 20.000 | 7% |
| Monounsaturated Fat | 4.612 | g | | |
| Polyunsaturated Fat | 0.936 | g | | |
| MFA 18:1, Oleic | 4.438 | g | | |
| PFA 18:2, Linoleic | 0.878 | g | | |
| PFA 18:3, Linolenic | 0.766 | g | | |
| PFA 20:5, EPA | 0.000 | g | | |
| PFA 22:6, DHA | 0.000 | g | | |
| Trans Fatty Acid | 0.075 | g | | |
| Sodium | 699.307 | mg | 2400.000 | 29% |
| Potassium | 1139.823 | mg | 3500.000 | 33% |
| Vitamin A (RE) | 45.153 | RE | | |
| Vitamin A (IU) | 21001.410 | IU | 5000.000 | 420% |
| Vitamin A (RAE) | 18.400 | μg | | |
| Beta-Carotene | 194.555 | μg | | |
| Alpha-Carotene | 11.343 | μg | | |
| Lutein (+Zeaxanthin) | 95.102 | μg | | |
| Beta-Cryptoxanthin | 41.028 | μg | | |
| Lycopene | 0.087 | μg | | |
| Vitamin C | 30.125 | mg | 60.000 | 50% |
| Calcium | 551.141 | mg | 1000.000 | 55% |
| Iron | 9.389 | mg | 18.000 | 52% |
| Vitamin D (ug) | 0.000 | μg | 10.000 | 0% |
| Vitamin D (IU) | 199.250 | IU | 400.000 | 50% |
| Vitamin E (mg) | 0.078 | mg | 20.000 | 0% |
| Vitamin E (IU) | 15.110 | IU | 30.000 | 50% |
| Alpha-Tocopherol | 0.381 | mg | | |
| Thiamin | 0.756 | mg | 1.500 | 50% |
| Riboflavin | 0.851 | mg | 1.700 | 50% |
| Niacin | 9.994 | mg | 20.000 | 50% |
| Pyridoxine (Vitamin B6) | 1.012 | mg | 2.000 | 51% |
| Folate (Total) | 200.749 | μg | 400.000 | 50% |
| Folate (DFE) | 2.919 | μg | | |
| Cobalamin (Vitamin B12) | 3.050 | μg | 6.000 | 51% |
| Biotin | 150.323 | μg | 300.000 | 50% |
| Pantothenic Acid | 5.065 | mg | 10.000 | 51% |
| Vitamin K | 40.179 | μg | 80.000 | 50% |
| Phosphorus | 757.914 | mg | 1000.000 | 76% |
| Iodine | 75.000 | μg | 150.000 | 50% |
| Magnesium | 205.145 | mg | 400.000 | 51% |
| Zinc | 7.707 | mg | 15.000 | 51% |
| Copper | 1.030 | mg | 2.000 | 51% |
| Manganese | 1.087 | mg | 2.000 | 54% |
| Selenium | 35.762 | μg | 70.000 | 51% |
| Fluoride | 0.002 | μg | | |
| Chromium | 0.061 | mg | 0.120 | 51% |
| Molybdenum | 38.500 | μg | 75.000 | 51% |
| Dietary Fiber, Total | 8.052 | g | 25.000 | 32% |
| Soluble Fiber | 0.226 | g | | |
| Insoluble Fiber | 2.271 | g | | |
| Crude Fiber | 0.272 | g | | |
| Sugar, Total | 4.532 | g | | |
| Glucose | 0.025 | g | | |
| Galactose | 0.002 | g | | |
| Fructose | 1.052 | g | | |
| Sucrose | 0.104 | g | | |
| Lactose | 0.000 | g | | |
| Maltose | 0.000 | g | | |
| Sugar Alcohol | 0.000 | g | | |
| Other Carbohydrate | 18.052 | g | | |
| Tryptophan | 303.813 | mg | | |
| Threonine | 965.990 | mg | | |
| Isoleucine | 1218.725 | mg | | |
| Leucine | 2062.106 | mg | | |
| Lysine | 1684.206 | mg | | |
| Methionine | 508.757 | mg | | |
| Cystine | 229.929 | mg | | |
| Phenylalanine | 1177.150 | mg | | |
| Tyrosine | 998.849 | mg | | |
| Valine | 1357.549 | mg | | |
| Arginine | 1630.893 | mg | | |
| Histidine | 621.853 | mg | | |
| Alanine | 843.507 | mg | | |
| Aspartic Acid | 2221.129 | mg | | |
| Glutamic Acid | 4709.236 | mg | | |
| Glycine | 743.956 | mg | | |
| Proline | 681.382 | mg | | |
| Serine | 1221.597 | mg | | |
| Moisture | 2.308 | g | | |
| Ash | 2.311 | g | | |
| Caffeine | 0.000 | mg | | |

DAILY VALUES/RDI - ADULT/CHILD

| Percentage of Kcals | |
|---|---|
| Protein | 33.0% |
| Carbohydrate | 41.5% |
| Fat, total | 25.5% |
| Alcohol | 0.0% |

The above formulations and nutrient analyses are but few of the myriad food compositions that can be developed under the subject invention, and do not limit but simply illustrate the invention. Other compositions would be apparent to those skilled in the field based on the totality of this disclosure.

What is claimed is:

1. A food composition comprising the following essential nutrients: water, choline, omega-3, omega-6, leucine, isoleucine, lysine, methionine, cysteine, phenylalanine, tyrosine, tryptophan, threonine, valine, histidine, arginine, vitamin A, vitamin C, calcium, iron, vitamin D, vitamin E, vitamin K, thiamin, riboflavin, niacin, vitamin B6, folate, vitamin B12, biotin, pantothenic acid, phosphorus, iodine, magnesium, zinc, selenium, copper, manganese, chromium, molybdenum, and chloride.

2. The food composition of claim 1 comprising approximately the following amounts of the essential nutrients:

| | |
|---|---|
| Water (mL) | 1-500 |
| Choline (mg) | 1-1,000 |
| Omega-3 (mg) | 1-5,000 |
| Omega-6 (mg) | 1-5,000 |
| Leucine, isoleucine, | 1-100 |
| lysine, methionine, | |
| cysteine, | |
| phenylalanine, | |
| tyrosine, tryptophan, | |
| threonine, valine, | |
| histidine, arginine | |
| (to yield dietary | |
| protein in grams) | |
| Vitamin A (IU) | 1,000-3,500 |
| Vitamin C (mg) | 15-45 |
| Calcium (mg) | 250-750 |
| Iron (mg) | 9 |
| Vitamin D (IU) | 100-400 |
| Vitamin E (IU) | 10-30 |
| Vitamin K (mcg) | 10-80 |
| Thiamin (mg) | 0.25-1.5 |
| Riboflavin (mg) | 0.4-1.7 |
| Niacin (mg) | 3-20 |
| Vitamin B6 (mg) | 0.5-2 |
| Folate (mcg) | 100-400 |
| Vitamin B12 (mcg) | 1-6 |
| Biotin (mcg) | 50-300 |
| Pantothenic acid (mg) | 1-10 |
| Phosphorus (mg) | 100-1,000 |
| Iodine (mcg) | 25-150 |
| Magnesium (mg) | 50-400 |
| Zinc (mg) | 1-15 |
| Selenium (mcg) | 10-70 |
| Copper (mg) | 0.25-2 |
| Manganese (mg) | 0.1-2 |
| Chromium (mcg) | 10-120 |
| Molybdenum (mcg) | 10-75 |
| Chloride (mg) | 500-3,400 |

3. The food composition of claim 2 comprising approximately the following amounts of the essential nutrients:

| | |
|---|---|
| Water (mL) | 300 |
| Choline (mg) | 100 |
| Omega-3 (gm) | 1,500 |
| Omega-6 (gm) | 1,500 |
| Leucine, isoleucine, | 25 |
| lysine, methionine, | |
| cysteine, | |
| phenylalanine, | |
| tyrosine, tryptophan, | |
| threonine, valine, | |
| histidine, arginine | |
| (as dietary protein in | |
| grams) | |
| Vitamin A (IU) | 2,500 |
| Vitamin C (mg) | 30 |
| Calcium (mg) | 500 |
| Iron (mg) | 9 |
| Vitamin D (IU) | 200 |
| Vitamin E (IU) | 15 |
| Vitamin K (mcg) | 40 |
| Thiamin (mg) | 0.75 |
| Riboflavin (mg) | 0.85 |
| Niacin (mg) | 10 |
| Vitamin B6 (mg) | 1 |
| Folate (mcg) | 200 |
| Vitamin B12 (mcg) | 3 |
| Biotin (mcg) | 150 |
| Pantothenic acid (mg) | 5 |
| Phosphorus (mg) | 500 |
| Iodine (mcg) | 75 |
| Magnesium (mg) | 200 |
| Zinc (mg) | 7.5 |
| Selenium (mcg) | 35 |
| Copper (mg) | 1 |
| Manganese (mg) | 1 |
| Chromium (mcg) | 60 |
| Molybdenum (mcg) | 37.5 |
| Chloride (mg) | 1,700 |

4. The food composition of claim 2 further comprising from 200-500 kcal.

5. The food composition of claim 4 comprising 350 kcal.

6. The food composition of claim 1 comprising one or more carbohydrate sources selected from the group of carbohydrate sources consisting of rice, brown sugar, white sugar, turbinado sugar, oats, apples, tomato, fructose, lactose, buttermilk, maltodextrin, corn starch, carrot, instant rice, red bell peppers, durum wheat flour, mushroom, potato, spinach, sweet corn, molasses, and beets.

7. The food composition of claim 1 comprising one or more protein sources selected from the group of protein sources consisting of textured vegetable protein, milk protein isolate, non-fat dry milk, milk protein (concentrate), whole milk powder, oat protein, mozzarella cheese, whey protein, yeast extract, red beans, chicken, parmesan cheese, dehydrated cheddar cheese, Vermont cheddar cheese, blue cheese, and peanut flour.

8. The food composition of claim 1 comprising one or more fat sources selected from the group of fat sources consisting of rice bran oil, butter, buttermilk, extra virgin olive oil, butter oil, sweet cream solids, ghee, flax, high-oleic safflower oil, and sesame oil.

9. The food composition of claim 1 comprising one or more fiber sources selected from the group of fiber sources consisting of hulled hemp seeds, oat fiber, cellulose gum and carrageenan.
